(19) United States
(12) Patent Application Publication
(10) Pub. No.: US 2019/0201376 A1
(43) Pub. Date: Jul. 4, 2019

(54) Method for Treating or Preventing Fatty Acid Binding Protein 3 Induced B-amyloid Aggregation Diseases

(71) Applicants: NATIONAL TAIWAN NORMAL UNIVERSITY, TAIPEI CITY (TW); ACADEMIA SINICA, TAIPEI CITY (TW)

(72) Inventors: Jung-Yaw LIN, Taipei City (TW); Su-Chang LIN, Taipei City (TW)

(21) Appl. No.: 16/207,295

(22) Filed: Dec. 3, 2018

(30) Foreign Application Priority Data
Dec. 28, 2017 (TW) 106146214

(51) Int. Cl. A61K 31/366; A61P 25/28

(52) U.S. Cl. CPC A61K 31/366; A61P 25/28

(57) ABSTRACT

A method for treating or preventing fatty acid binding protein 3 induced β-amyloid aggregation diseases is disclosed, comprising administering a pharmaceutical composition to a subject in need, where in the pharmaceutical composition comprises artemisinin.

### CROSS REFERENCE TO RELATED APPLICATION

This application claims the benefits of the Taiwan Patent Application Serial Number 106146214, filed on Dec. 28, 2017, the subject matter of which is incorporated herein by reference.

### BACKGROUND OF THE INVENTION

### 1. Field of the Invention

The present invention relates to a method for treating or preventing abnormal proteins aggregation diseases with a pharmaceutical composition. More specifically, the present invention relates to a method for treating or preventing abnormal β-amyloid aggregation diseases, and the pharmaceutical composition comprises artemisinin.

### 2. DESCRIPTION OF RELATED ART

Alzheimer's disease (AD) is the most prevalent form of dementia in elderly patients causing neurodegeneration. The progressive cognitive decline and memory loss are usually observed in AD patients, and health expenditures and costs of care are high and expensive for AD patients.

Nowadays, some drugs are proved having efficacy of improving cognitive impairment. Currently, two kinds of drugs have been proved by the U.S. Food and Drug Administration, one is cholinesterase inhibitors including rivastigmine, donepezil and galantamine, and the other one is N-methyl-D-aspartate (NMDA) receptor antagonist such as memantine. Except for the administration of drugs for improving cognitive impairment, other suitable drugs also have to be administered to AD patients with other symptoms derived from AD such as depression and sleeplessness.

The worldwide populations with AD are gradually increased. Therefore, it is desirable to provide a method or a pharmaceutical composition for treating β-amyloid aggregation diseases, which can be used to treat neurodegenerative diseases such as AD to further delay disease progression and improve patients' quality of life.

### SUMMARY OF THE INVENTION

The object of the present invention is to provide a pharmaceutical composition for treating or preventing fatty acid binding protein 3 induced β-amyloid aggregation diseases, wherein the pharmaceutical composition comprises artemisinin.

Another object of the present invention is to provide a pharmaceutical composition for inhibiting fatty acid binding protein 3 induced β-amyloid aggregation diseases, wherein the pharmaceutical composition comprises artemisinin.

Another object of the present invention is to provide a method for treating or preventing fatty acid binding protein 3 induced β-amyloid aggregation diseases with the pharmaceutical composition of the present invention, wherein the pharmaceutical composition comprises artemisinin.

A further object of the present invention is to provide a use of the pharmaceutical composition of the present invention for manufacturing a drug of fatty acid binding protein 3 induced β-amyloid aggregation diseases, wherein the pharmaceutical composition comprises artemisinin. In addition, another object of the present invention is to provide a pharmaceutical composition for inhibiting fatty acid binding protein 3 induced β-amyloid aggregation, wherein the pharmaceutical composition comprises artemisinin.

Yet another object of the present invention is to provide a method for inhibiting fatty acid binding protein 3 induced β-amyloid aggregation in a subject with the pharmaceutical composition of the present invention, wherein the pharmaceutical composition comprises artemisinin.

A further object of the present invention is to provide a use of the pharmaceutical composition of the present invention for manufacturing a drug for inhibiting fatty acid binding protein 3 induced β-amyloid aggregation, wherein the pharmaceutical composition comprises artemisinin.

In the present invention, the fatty acid binding protein 3 induced β-amyloid aggregation disease is not limited. Preferably, the fatty acid binding protein 3 induced β-amyloid aggregation disease is a neurodegenerative disease associated with β-amyloid aggregation. More preferably, the fatty acid binding protein 3 induced β-amyloid aggregation disease is Alzheimer's disease.

In the pharmaceutical composition of the present invention, the concentration of the artemisinin is not limited, and may be adjusted depending on disorder severity or complementary medicines. In one preferred embodiment of the present invention, the concentration of the artemisinin may, by way of example and not limitation, be in a range from 0.1 μM to 10 μM based on a total weight of the pharmaceutical composition.

In one preferred embodiment of the present invention, the pharmaceutical composition may prevent or treat the fatty acid binding protein 3 induced β-amyloid aggregation disease by inhibiting fatty acid binding protein 3 (FABP3).

In another preferred embodiment of the present invention, the pharmaceutical composition may inhibit β-amyloid aggregation by inhibiting fatty acid binding protein 3 (FABP3).

Moreover, yet another object of the present invention is to provide a method for treating or preventing an fatty acid binding protein 3 induced β-amyloid aggregation disease with the pharmaceutical composition of the present invention comprising artemisinin, wherein the artemisinin combines with fatty acid binding protein 3 (FABP3).

In the present invention, the fatty acid binding protein 3 induced β-amyloid aggregation disease is not limited. Preferably, the fatty acid binding protein 3 induced β-amyloid aggregation disease is a neurodegenerative disease associated with β-amyloid aggregation. More preferably, the fatty acid binding protein 3 induced β-amyloid aggregation disease is Alzheimer's disease.

In the pharmaceutical composition, the concentration of artemisinin is not limited, and may be adjusted depending on disorder severity or complementary medicines. In one preferred embodiment, the concentration of artemisinin may, by way of example and not limitation, be in a range from 0.1 μM to 10 μM based on a total weight of the pharmaceutical composition.

In the present invention, the pharmaceutical composition may further comprise: at least one pharmaceutically acceptable carrier, diluent, or excipient.

In one preferred embodiment of the present invention, the pharmaceutical composition may prevent or treat the fatty acid binding protein 3 induced β-amyloid aggregation disease by inhibiting fatty acid binding protein 3 (FABP3).

The pharmaceutical composition may further comprise: at least one pharmaceutically acceptable carrier, diluent, or excipient. For example, the compound may be encapsulated into liposome to facilitate delivery and absorption. Alternatively, the compound may be diluted with aqueous suspension, dispersion or solution to facilitate injection. Or, the compound may be prepared in a form of a capsule or tablet for storage and carrying. In addition, an effective concentration of the compound of the artemisinin may be changed according to administration, use of excipient, or co-use with other active agents; and a person skilled in the art may adjust the concentration of the artemisinin in the pharmaceutical composition or the dose of the pharmaceutical composition to achieve the purpose of obtaining desired curative effect.

More specifically, the artemisinin of the present invention may be formulated in a solid or liquid form. The solid formulation form may include, but is not limited to, powders, granules, tablets, capsules and suppositories. The solid formulation may comprise, but is not limited to, excipients, flavoring agents, binders, preservatives, disintegrants, glidants and fillers. The liquid formation form may include, but is not limited to, water, solutions such as propylene glycol solution, suspensions and emulsions, which may be prepared by mixing with suitable coloring agents, flavoring agents, stabilizers and viscosity-increasing agents.

For example, a powder formulation may be prepared by simply mixing the artemisinin of the present invention with suitable pharmaceutically acceptable excipients such as sucrose, starch and microcrystalline cellulose. A granule formulation may be prepared by mixing the artemisinin of the present invention with suitable pharmaceutically acceptable excipients and/or suitable pharmaceutically acceptable binders such as polyvinyl pyrrolidone and hydroxypropyl cellulose, followed by wet granulation method using a solvent such as water, ethanol and isopropanol, or dry granulation method using compression force. Also, a tablet formulation may be prepared by mixing the granule formulation with suitable pharmaceutically acceptable glidants such as magnesium stearate, followed by tableting using a tablet machine. Hence, a person skilled in the art may appropriately choose suitable formulation according to his/her needs.

To implement the method according to the present invention, the above pharmaceutical composition may be administered via oral administering, parenteral administering, inhalation spray administering, topical administering, rectal administering, nasal administering, sublingual administering, vaginal administering, or implanted reservoir, and so on. The term “parenteral” used here refers to subcutaneous injection, intradermal injection, intravenous injection, intramuscular injection, intra-articular injection, intra-arterial injection, joint fluid injection, intrathoracic injection, intrathecal injection, injection at morbid site, and intracranial injection or injection technique.

The term “treat” or “treating” used herein refers to the treatment of a disease that alleviates, mitigates, or ameliorates: at least one symptom or condition of a disease; inhibits a disease or condition; prevents or mitigates the progression of a disease; recovers a disease or condition; mitigates the physiological condition caused by a disease; halts a disease symptom or physiological condition.

Other objects, advantages, and novel features of the invention will become more apparent from the following detailed description when taken in conjunction with the accompanying drawings.

### BRIEF DESCRIPTION OF THE DRAWINGS

FIG. 1 shows levels of Aβ and FABP3 in 3×Tg AD transgenic mice at different ages analyzed by western blot according to one preferred embodiment of the present invention.

FIG. 2A shows the correlation between FABP3 and Aβ₄₂ oligomer and the efficacy of artemisinin inhibiting oligomerization of Aβ₄₂ analyzed by western blot according to one preferred embodiment of the present invention.

FIG. 2B shows a quantization table of FIG. 2A by using ImageJ.

FIG. 3A shows the result of cell viability assay after treating artemisinin according to one preferred embodiment of the present invention.

FIG. 3B shows the efficacy of artemisinin inhibiting oligomerization of Aβ₄₂, which is induced by FABP3, by using western blot according to one preferred embodiment of the present invention.

FIG. 4 shows the structure of human. FABP3 with artemisinin according to one preferred embodiment of the present invention.

FIG. 5 shows the animal behavior after treating artemisinin in 3×Tg AD transgenic mice according to one preferred embodiment of the present invention.

### DETAILED DESCRIPTION OF THE PREFERRED EMBODIMENT

### [Example 1] Western Blot Analysis of Aβ and FABP3 in the Cerebral Tissues of 3×Tg AD Transgenic Mice

Animals

In the present embodiment, 3×Tg AD transgenic mice harboring human PS1_{M146V}, human APP_{Swe}, and human tau_{P301L} were used, which were obtained from the Jackson Laboratory (004807). In the present embodiment, the 4-, 6-, and 24-month-old mice (n=3 per group) were respectively anesthetized with urethane (1.5 mg/kg) and transcardially perfused with physiological saline. Afterwards, the left hemispheres of brain tissues were collected in cold RIPA buffer supplemented with cOmplete™ Protease Inhibitor Cocktail (Sigma-Aldrich), homogenized by 22G and 26G (32 mm and 13 mm) needles (TERUMO Needle, NEOLUS) on ice, and then stored at −80° C.

Western Blot Analysis

The protein concentrations were detected with BCA protein assay kit (Thermo scientific), and each equal amount of protein samples (40 μg) was resolved on 12.5% SDS-PAGE. After electrophoresis and semi-thy blotting, the PVDF membrane was blocked with 5% non-fat milk solution in TBST at room temperature for one hour or overnight at 4° C., then, washed thrice with TBST for five minutes. Subsequently; the membrane was incubated with anti-Aβ (6E10), FABP3 or GAPDH primary antibodies in dilute buffer overnight at 4° C. In addition, GAPDH was used as the loading control. After washing in TBST buffer for five min thrice, the membrane was treated with HRP-conjugated secondary antibodies at room temperature for one hour. The proteins were visualized by using an ECL detection reagent (Millipore) and detected with an ImageQuest™ LAS-4000 (Fujifilm Co., Tokyo, Japan). The expression of protein was quantified by ImageJ (National Institute of Health, USA).

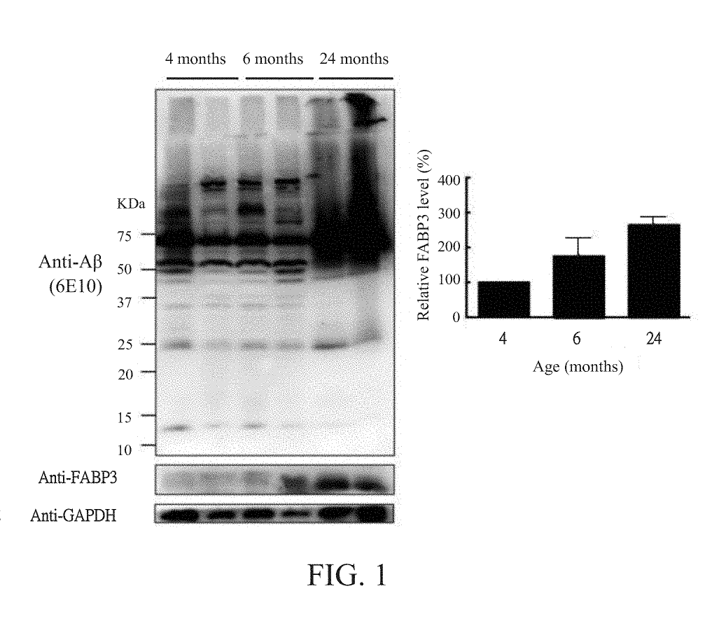

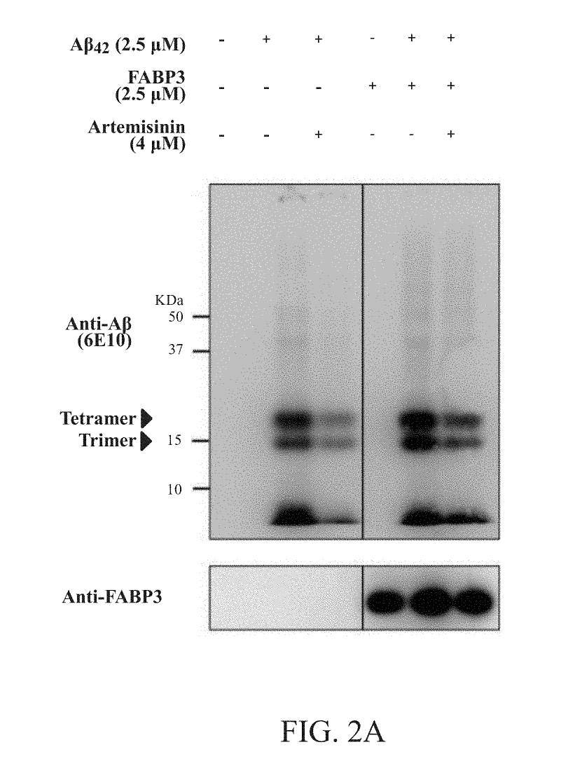

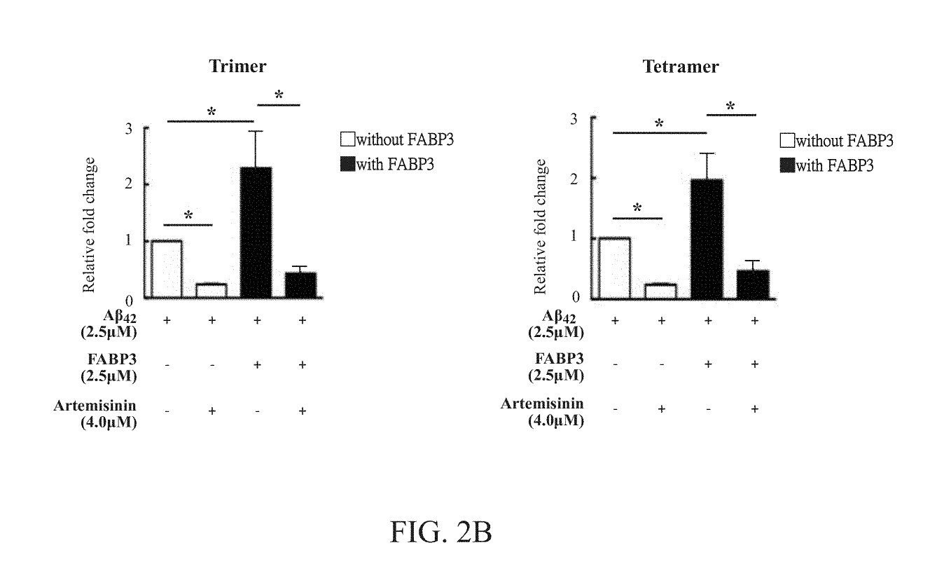

FIG. 1 shows levels of Aβ and FABP3 in 3×Tg AD transgenic mice at different ages analyzed by western blot. As shown in FIG. 2, a 163% increase in FABP3 expression was found in the 24-month-old mice, related to the formation of Aβ oligomers from 4 months to 24 months of age, implying that FABP3 plays a role in AD pathogenesis.

### [Example 2] Western Blot Analysis for the Correlation Between FABP3 and Aβ, and the Efficacy of Artemisinin Inhibiting the Oligomerization of Aβ₄₂

Aβ₄₂ Oligomer Preparation

Synthetic Aβ₄₂ peptides (AnaSpec) were solubilized in hexafluoroisopropanol (HFIP), and then HFIP was allowed to be evaporated completely under nitrogen flow. Aβ₄₂ peptides were resuspended using phosphate buffered saline (PBS) to 65 mM, and were incubated to assemble oligomers at room temperature for 24 hours prior to use. The oligomeric status of AP was verified by sodium dodecyl sulfate polyacrylamide gel electrophoresis (SDS-PAGE).

FIG. 2A shows the correlation between FABP3 and Aβ₄₂ oligomer and the efficacy of artemisinin inhibiting oligomerization of Aβ₄₂ analyzed by western blot. As shown in FIG. 2A, left panel is oligomers (2.5 μM) incubated with or without artemisinin (Sigma-Aldrich) (4 μM) at room temperature for 24 hours; right panel is recombinant human FABP3 protein (Cayman Chemical) (2.5 μM) pre-incubated with or without artemisinin (4 μM) at room temperature for four hours, and then incubated with Aβ₄₂ oligomers (2.5 μM) for 24 hours. Afterwards, the oligomerization of Aβ₄₂ was analyzed using western blot analysis and detected with 6E10 antibody.

FIG. 2B shows a quantization table of FIG. 2A by using ImageJ (National Institute of Health, USA). As shown in FIG. 2B, the levels of Aβ₄₂ trimer and tetramer detected by 6E10 antibody significantly increased in the presence of FABP3 by 129% and 96% (p<0.05), respectively. Furthermore, the preincubation of artemisinin (4 μM) with FABP3 (2.5 μM) for four hours remarkably decreased Aβ₄₂ trimer and tetramer induced by FABP3 by 144% and 156% (p<0.05), respectively.

In the present embodiment, it was examined that whether artemisinin, isolated from Chinese herb Artemisia annua, is a potential anti-AD agent by targeting FABP3. These data suggest that Aβ₄₂ oligomerization induced by FABP3 could be prevented mitigated by artemisinin, and thus it can be used to prepare an anti-AD agent.

### [Example 3] Establishment of Stably Transfected Human FABP3-SH-SY5Y Cell Model and Cell Viability Assay

Cell Culture

Human neuroblastoma SH-SY5Y cell line was obtained from American Type Culture Collection (ATCC® CRL-2266™) and maintained in Dulbecco's Modified Eagle Medium with nutrient mixture F-12 (DMEM/F12; Invitrogen) media supplemented with 10% FBS, 100 U/mL penicillin and 100 μg/mL streptomycin at 37° C. in a 5% CO₂ humidified incubator.

FABP3 cDNA Construction and the Establishment of Stably Transfected Cell Line

Human FAPB3 cDNA, which was reverse transcribed from the total RNA of SH-SY5Y cells, was amplified by polymerase chain reaction (PCR) using primers (5′-CACCATGGTGGACGCTITCCTG and 5′-TGCCTCTTCTCATAAGTG) and then cloned into pcDNA™3.1D/V5-His-TOPO vector (Invitrogen). SH-SY5Y cells stably expressing human FABP3 were obtained using lipofectamine transfection with selection via cultivation in DMEM/F12 medium (Gibco) containing 500 g/mL G418 (Sigma-Aldrich).

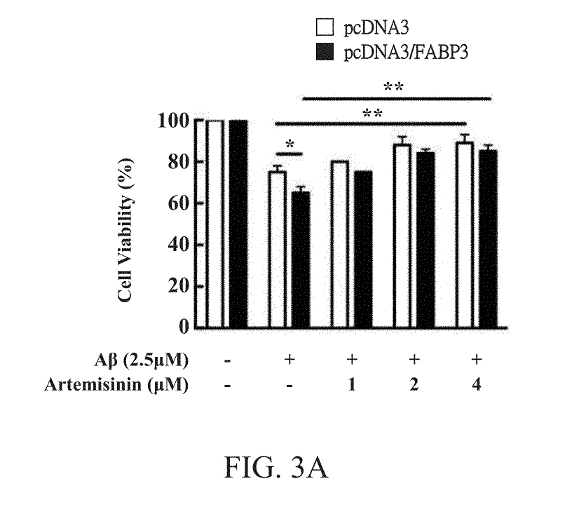

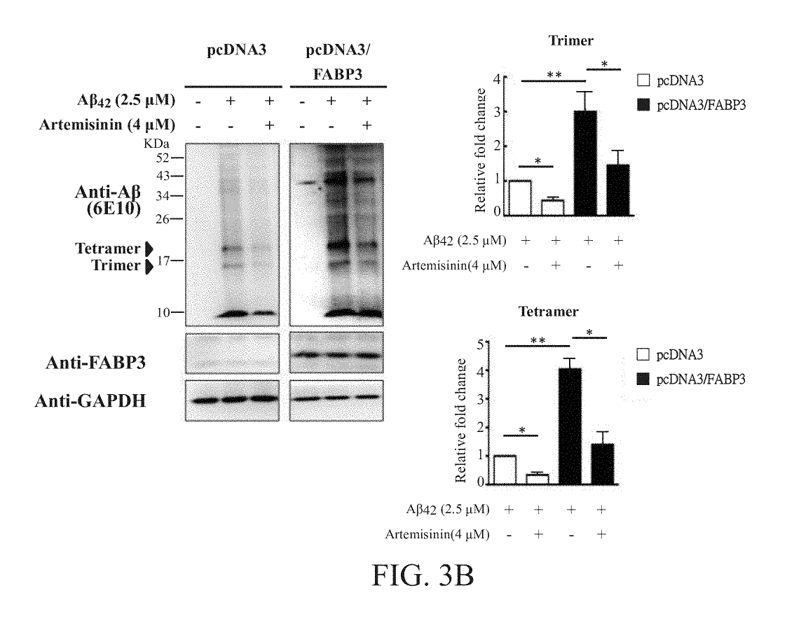

FIG. 3A shows the result of cell viability assay. As shown in FIG. 3A, SH-SY5Y cells (2×10⁴) transfected with vacant vector (i.e. pcDNA3 group) and SH-SY5Y cells (2×10⁴) transfected with human FABP3 (i.e. pcDNA3/FABP3 group) were respectively seeded in 96-well plates, pretreated with artemisinin (Sigma-Aldrich) for 4 hours, treated with Aβ₄₂ (2.5 μM) for 24 hours, and washed with PBS before the addition of MTT solution (5 mg/ml, Sigma-Aldrich). Afterwards, purple formazan crystals formed by reduction of MTT in the mitochondria of living cells were dissolved in 100 μl solution containing 10% SDS and 0.01N HCl. The absorbance was measured at 570 nm with an ELISA reader (pQuant; BioTek Instruments, Inc).

As shown in FIG. 3A, the overexpression of FABP3 significantly decreased the cell viability from 75%, which was caused by Aβ₄₂ (2.5 μM) alone, to 65% (p<0.01). In addition, pretreating the cells with artemisinin (0-4 μM) for four hours may significantly increase the cell viability from 65% to 85% (p<0.01), as shown in FIG. 3A.

The present embodiment established a stably transfected human FABP3-SH-SY5Y cell model, and examined whether artemisinin can inhibit the Aβ₄₂ cell cytotoxicity induced by FABP3. These data suggest that artemisinin may effectively reduce the Aβ₄₂ cell cytotoxicity induced by FABP3.

### [Example 4] Western Blot Analysis for the Correlation Between FABP3 and Aβ₄₂ in Stably Transfected Human FABP3-SH-SY5Y and Normal Human FABP3-SH-SY5Y Cell, and the Efficacy of Artemisinin Inhibiting the Oligomerization of Aβ₄₂

Cell Culture

Human neuroblastoma SH-SY5Y cell line was obtained from American Type Culture Collection (ATCC® CRL-2266™) and maintained in Dulbecco's Modified Eagle Medium with nutrient mixture F-12 (DMEM/F12; Invitrogen) media supplemented with 10% FBS, 100 U/mL penicillin and 100 μg/mL streptomycin at 37° C. in a 5% CO₂ humidified incubator.

FABP3 cDNA Construction and the Establishment of Stably Transfected Cell Line

Human FAPB3 cDNA, which was reverse transcribed from the total RNA of SH-SY5Y cells, was amplified by polymerase chain reaction (PCR) using primers (5′-CACCATGGTGGACGCTTTCCTG and 5′-TGCCTCTrTCTCATAAGTG) and then cloned into pcDNA™3.1D/V5-His-TOPO vector (Invitrogen). SH-SY5Y cells stably expressing human FABP3 were obtained using lipofectamine transfection with selection via cultivation in DMEM/F12 medium (Gibco) containing 500 μg/mL G418 (Sigma-Aldrich).

FIG. 3B shows the efficacy of artemisinin inhibiting oligomerization of Aβ₄₂, which is induced by FABP3, by using western blot. As shown in FIG. 3B, SH-SY5Y cells (106) transfected with vacant vector (i.e. pcDNA3 group) and human FABP3-SH-SY5Y cells (106) (i.e. pcDNA3/FABP3 group) were respectively seeded in 6 cm plates, pretreated with artemisinin (Sigma-Aldrich) (0 μM or 4 μM) for 4 hours, and treated with Aβ₄₂(2.5 μM) for 24 hours. Then, the stably transfected human FABP3-SH-SY5Y cells were washed with PBS and lysed with radioimmunoprecipitation (RIPA) buffer supplemented with cOmplete™ Protease Inhibitor Cocktail (Sigma-Aldrich). The samples with equal amounts of total protein (50 μg) were fractionated by SDS-PAGE and electrotransferred to a polyvinylidene difluoride (PVDF) membrane (Millipore). The membrane was blocked for one hour at room temperature with 10% nonfat milk in TBST buffer (20 mM Tris-HCl, 150 mM NaCl, and 0.1% Tween 20); thereafter, the membrane was incubated with 6E10 (BioLegend), FABP3 (Santa Cruz) or GAPDH (Santa Cruz) antibodies. Binding of the primary antibodies was followed by incubation for one hour at room temperature with the appropriate secondary antibodies conjugated to horseradish peroxidase. The signals were visualized using the enhanced chemiluminescence system (ECL; Millipore).

As shown in FIG. 3B, in the stably transfected human FABP3-SH-SY5Y cells, the levels of Aβ₄₂ trimer and tetramer were significantly increased by 200% and 304%, respectively. Noticeably, artemisinin (4 μM) effectively decreased Aβ₄₂ trimer and tetramer induced by FABP3 overexpression by 78% and 87%, respectively.

The results from Example 3 and Example 4 indicate that artemisinin may effectively attenuate the cytotoxicity of Aβ₄₂ induced by FABP3 and reduce Aβ₄₂ trimer and tetramer.

### [Example 5] Ligand Docking Experiments to Analyze Molecular Interactions Between FABP3 and Artemisinin

Ligand Docking Experiment

Three coordinates of FABP3 in complexes with 6-chloro-2-methyl-4-phenyl-quinoline-3-carboxylic acid (5M8, from PDBID: 5HZ9), oleic acid (OLA, PDBID: 5CE4), and 8-anilinonaphthalene-1-sulfonic acid (2AN, from PDBID:3WBG), respectively, were downloaded from the PDB database. The ligand and water molecules were removed from docking. The FABP3 coordinates were individually uploaded to the SwissDock web server, together with the ligand coordinate of artemisinin obtained from the ZINC database with the entry number 8143788. After docking, the results that contained the molecule of artemisinin with a variety of orientations were retrieved from the server. The results were inspected and the top docking results were shown in the figures using the UCSF Chimera package. To determine the feasibility of the docking experiments, the coordinates of 5M8, OLA, and 2AN were also used respectively for docking.

Generation of the Electrostatic Potential Surface and Molecular Graphics

Molecular graphics images were obtained using the UCSF Chimera package from the Computer Graphics Laboratory, University of California, San Francisco. To generate the electrostatic potential surface, the models of FABP3 were uploaded to a web service to generate the PQR files by using the PDB2PQR tool, which were subsequently used to generate the DX files by using the APBS tool, as implemented in the Chimera Image Tutorial. The resulting electrostatic potential map was used to color the molecular surfaces in the Chimera. Molecular superimposition was also conducted using the Chimera package.

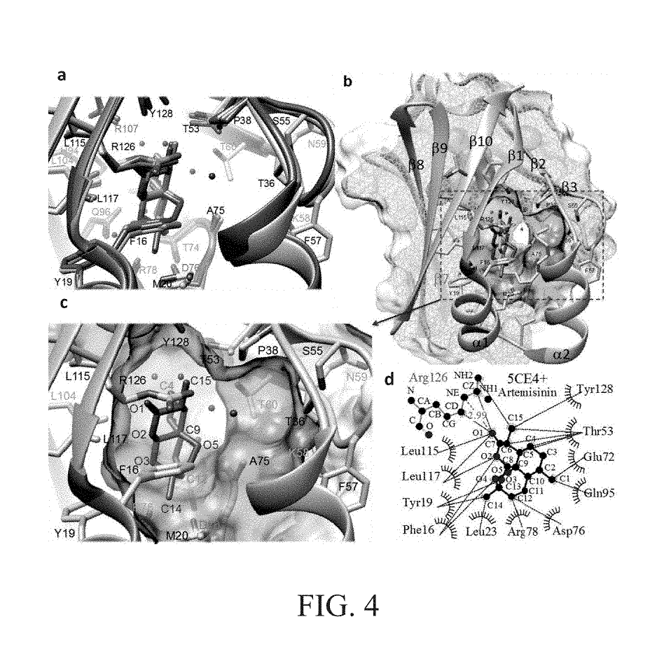

FIG. 4 shows the structure of human FABP3 with artemisinin. FIG. 4a shows the superimposition of the human FABP3 crystal structures docked with artemisinin. The FABP3 structures from PDB: 5CE4, PDB: 5HZ9, and PDB: 3WBG, were superimposed using the UCSF Chimera package and shown in pink, blue, and green, respectively, with docked artemisinin shown in red, blue, and green. The side chains of some residues in the substrate-binding pocket were shown.

In addition, the molecular interactions between human FABP3 and artemisinin were predicted using the SwissDock web server. In each docking experiment, many binding modes had been generated and clustered. As shown in FIG. 4a, the clustering results showed that the molecule of artemisinin in the most favorable cluster was docked within the substrate-binding pocket of FABP3 with an orientation similar to those in the other experiments, suggesting that the most favorable binding mode was consistent.

FIG. 4b shows a clipped and transparent surface representation of the FABP3 structure (PDB: 5CE4, in pink ribbons) showing an oleic acid molecule (in pink sticks) and docked artemisinin (in red sticks) in the substrate-binding pocket. The green arrow and a dashed circle respectively indicated two entries of the substrate-binding pocket. The clipped surface was capped with a mesh in cyan. The clipped and transparent surface of the substrate-binding pocket of FABP3 was colored with electrostatic potential, calculated by APBS, and plotted at ±10 kT/e (red, negative; blue, positive). The helices and some beta-strands were labeled. The molecule of artemisinin preferentially resided at the bottom of the substrate-binding pocket, which was away from both entries to it and with an estimated Gibbs free energy (ΔG) ranging from −7.51 to −7.63 kcal/mol and FullFitness value ranging from −774.94 to −895.10 kcal/mol FIG. 4c shows a close-up view of the substrate-binding pocket in FIG. 4b, with docked artemisinin. For clarity, the clipped surface was not capped in order to show the residues around the substrate-binding pocket. Some atoms of artemisinin were labeled in red. The red dots represent the water molecules found in the crystal structure.

FIG. 4d, generated by the LigPlot+ suite, shows the predicted intermolecular interactions between FABP3 and artemisinin. The green dashed lines indicated the hydrogen bonds or salt bridges between FABP3 and artemisinin. The red dashed lines indicated the hydrophobic contacts or van der Waals interactions between FABP3 and artemisinin.

As shown in FIG. 4b to 4d, in all three docking models, ten FABP3 residues were found to interact with artemisinin, including F16 and Y19 on al, E72 on 135, D76 and R78 on loop 15-16, Q95 on 3, L115 and Lll7 on 139, and R126 and Y128 on 1310. In addition, L23, T53, A75, and L104 may also interact with artemisinin, although their interactions were not found in all three docking models. In the docking models, FABP3 residues R126 and potentially Y128 were found to establish a hydrogen bond with artemisinin oxygen atom O1. F16, Y19, L115, L117, and R126 interacted with artemisinin oxygen atoms O1, O2, and O3 via van der Waals interactions. T53, E72, A75, D76, R78, Q95, and Y128 also interacted with the artemisinin carbon atoms also via van der Waals interactions. Y19, L23, T53, A75, and L106 were predicted to establish hydrophobic interactions with artemisinin.

### [Example 6] Examination of the Effect of Artemisinin on Improvement of Memory Impairments and Cognition Loss in Alzheimer's Disease Mice by Animal Behavior Test

Animals

In the present embodiment, 3×Tg AD transgenic mice were obtained from the Jackson Laboratory (004807), and wild-type C57BL/6 mice were purchased from the National Laboratory Animal Center. The body weights of the animals were measured every eight weeks from 12^{th} to 36^{th} week-old. The animals were i.p. administered with pure water or artemisinin (1 mg/kg) every other day from 12 weeks to 36 weeks of age; the WT-W group was water vehicle-treated wild-type mice; the AD-W group was water vehicle-treated 3×Tg AD transgenic mice; and AD-R group was artemisinin-treated 3×Tg AD transgenic mice. The animals were examined by using behavioral tasks, including Morris water maze test, spontaneous alternation behavior Y-maze test, and novel object recognition task test, when they were 36-week-old.

Morris Water Maze Test

To evaluate whether artemisinin ameliorates the spatial learning and memory deficits, the Morris water maze test was performed. For Morris water maze test, the 36-week-old mice (n=6 per group) administered with aforementioned treatment were given swim training in a white circular pool (100 cm in diameter and 35 cm in height) for 60 seconds in the absence of the platform before the experiment, and then four training trials per day for four consecutive days, with an inter-trial interval of 15 minutes. After the last trail, mice were subjected to the probe test that the platform was removed, and the time spent in quadrants was recorded by a computer-controlled system.

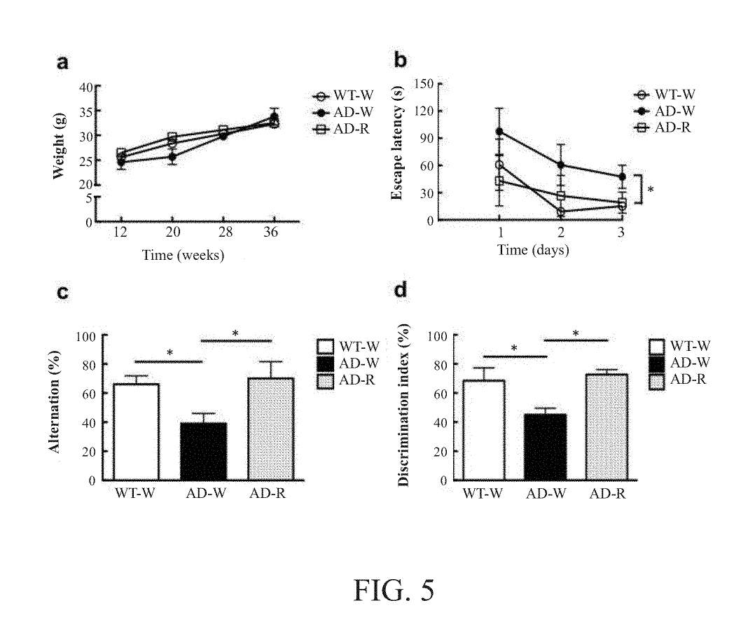

As shown in FIG. 5b, the 3×Tg AD mice treated with artemisinin spend significantly less time in finding the hidden platform (p<0.05).

Spontaneous Alternation Behavior Y-Maze Test

The spontaneous alternation behavior Y-maze test was used to estimate short-term memory. The Y-maze was a three-arm maze (30 cm long and 5 cm wide with 12 cm in height) with equal angles and the arms were labeled A, B, and C. The 36-week-old mice (n=6 per group) administered with aforementioned treatment were initially placed within one arm, and the number of arm entries and the number of alternations were recorded for eight min period for each mouse. Between each trial, the arms of Y-maze were cleaned by 70% ethanol to remove odors and residues. The percentage of alternation was calculated by the following equation: Alternation (%)=[(Number of alternation)/(Total arm entries−2)]×100.

As shown in FIG. 5c, the spatial working memory estimated by Y maze showed that a significant reduction of spontaneous alternation rate was observed in 3×Tg AD mice, whereas artemisinin administration restored spatial working memory to the extent similar as that of the wild-type group

Novel Object Recognition Test

The novel object recognition was conducted to examine whether artemisinin can restore recognition memory, which involves the frontal cortex, entorhinal cortex and hippocampus; it was used to evaluate the inborn tendency of the rodents to explore novel objects rather than familiar ones. During the first two days, mice were habituated to open field (50×40 cm, with 22 cm in height) for two sessions of 10 min each day in order to be familiar with the apparatus. On the third day, each mouse was submitted to a five min sample phase that two identical objects A were placed in the opposite corner of the open field arena, with the distance of 10 cm from the walls. After a five min delay, the mouse was removed and a familiar object was replaced with a novel one B in the same location. During the test phase, the mouse was placed back in the arena and exposed to two objects. The time spent exploring the objects (T_{A} and T_{B}, respectively) was defined as the distance from nose to object within 1-2 cm or/and touching it with the nose and forepaws. After each session, arena and objects were cleaned with 70% ethanol to prevent the olfactory cues. The discrimination index was calculated as percentage ratio of T_{B}/(T_{A}+T_{B})×100. A discrimination index of higher than 50% represents good cognitive performance.

As shown in FIG. 5d, compared to the wild-type group, the discrimination index for 3×Tg AD mice group showed a significant impairment (p<0.05). In contrast, 3×Tg AD mice treated with artemisinin significantly restored this phenotype with 56% increment in the discrimination index (p<0.05)

Taken together, these data suggest that artemisinin may be able to improve memory impairments and cognition loss in AD mice.

Although the present invention has been explained in relation to its preferred embodiment, it is to be understood that many other possible modifications and variations can be made without departing from the spirit and scope of the invention as hereinafter claimed.

What is claimed is:

1. A method for treating or preventing a fatty acid binding protein 3 (FABP3) induced β-amyloid aggregation disease, comprising administering a pharmaceutical composition comprising artemisinin.

2. The method according to claim 1, wherein the fatty acid binding protein 3 induced β-amyloid aggregation disease is a neurodegenerative disease associated with β-amyloid aggregation.

3. The method according to claim 1, wherein the fatty acid binding protein 3 induced β-amyloid aggregation disease is Alzheimer's disease.

4. The method according to claim 1, wherein a concentration of the artemisinin is in a range from 0.1 μM to 10 μM based on a total weight of the pharmaceutical composition.

5. The method according to claim 1, wherein the pharmaceutical composition further comprises: at least one pharmaceutically acceptable carrier, a diluent, or an excipient.

6. The method according to claim 1, wherein the pharmaceutical composition prevents or treats the fatty acid binding protein 3 induced β-amyloid aggregation disease by inhibiting fatty acid binding protein 3 (FABP3).

7. A method for inhibiting a fatty acid binding protein 3 induced β-amyloid aggregation disease, comprising administering a pharmaceutical composition comprising artemisinin.

8. The method according to claim 7, wherein the fatty acid binding protein 3 induced β-amyloid aggregation disease is a neurodegenerative disease associated with β-amyloid aggregation.

9. The method according to claim 7, wherein the fatty acid binding protein 3 induced β-amyloid aggregation disease is Alzheimer's disease.

10. The method according to claim 7, wherein a concentration of the artemisinin is in a range from 0.1 μM to 10 μM based on a total weight of the pharmaceutical composition.

11. The method according to claim 7, wherein the pharmaceutical composition further comprises: at least one pharmaceutically acceptable carrier, a diluent, or an excipient.

12. The method according to claim 7, wherein the pharmaceutical composition inhibits the fatty acid binding protein 3 induced β-amyloid aggregation disease by inhibiting fatty acid binding protein 3 (FABP3).

13. A method for treating or preventing a fatty acid binding protein 3 induced β-amyloid aggregation disease, comprising administering a pharmaceutical composition comprising artemisinin, wherein the artemisinin combines with fatty acid binding protein 3 (FABP3).

14. The method according to claim 13, wherein the fatty acid binding protein 3 induced β-amyloid aggregation disease is a neurodegenerative disease associated with β-amyloid aggregation.

15. The method according to claim 13, wherein the fatty acid binding protein 3 induced β-amyloid aggregation disease is Alzheimer's disease.

16. The method according to claim 13, wherein a concentration of the artemisinin is in a range from 0.1 μM to 10 μM based on a total weight of the pharmaceutical composition.

17. The method according to claim 13, wherein the pharmaceutical composition further comprises: at least one pharmaceutically acceptable carrier, a diluent, or an excipient.

18. The method according to claim 13, wherein the pharmaceutical composition prevents or treats the fatty acid binding protein 3 induced β-amyloid aggregation disease by inhibiting fatty acid binding protein 3 (FABP3).

19. A method for inhibiting fatty acid binding protein 3 induced β-amyloid aggregation, comprising administering a pharmaceutical composition comprising artemisinin to a subject in need.


SEQUENCE LISTING (2 sequences)

SEQ ID NO: 1 - DNA, 22 bp, Artificial
  Primer
caccatggtggacgctttcctg

SEQ ID NO: 2 - DNA, 19 bp, Artificial
  Primer
tgcctctttctcataagtg